(19) United States
(12) Patent Application Publication
(10) Pub. No.: US 2026/0069197 A1
(43) Pub. Date: Mar. 12, 2026

(54) Sexual health capacity measurement and analysis system, method, and device

(71) Applicant: Beihang University, Beijing (CN)

(72) Inventors: Xing CHEN, Beijing (CN); Hao CHEN, Beijing (CN)

(21) Appl. No.: 18/961,743

(22) Filed: Nov. 27, 2024

(30) Foreign Application Priority Data
Nov. 28, 2023 (CN) 202311606207.9

(51) Int. Cl. A61B 5/00; A61B 5/11

(52) U.S. Cl. CPC A61B 5/4368; A61B 5/1114; A61B 5/4393; A61B 5/725; A61B 5/002; A61B 5/6823; A61B 5/6831; A61B 2562/0219

(57) ABSTRACT

A sexual health capacity measurement and analysis system, method, and device are provided. The method includes: acquiring a relative acceleration of waist movements of a male and a female; acquiring a start time and an end time of sexual activity to obtain a duration; acquiring a thrust count, a thrust distance, and a thrust frequency of the male, and/or a thrust count, a thrust distance, and a thrust frequency of the female; obtaining position data based on the relative acceleration of the waist movements of the male and the female; and inputting the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

### CROSS REFERENCE TO RELATED APPLICATION

This patent application claims the benefit and priority of Chinese Patent Application No. 202311606207.9, filed with the China National Intellectual Property Administration on Nov. 28, 2023, the disclosure of which is incorporated by reference herein in its entirety as part of the present application.

### TECHNICAL FIELD

The present disclosure relates to the technical field of medical devices, and in particular, to a sexual health capacity measurement and analysis system, method, and device.

### BACKGROUND

Premature ejaculation is a common type of male dysfunction, with a high incidence rate. Approximately 75% of men experience symptoms of premature ejaculation at some point in their lives.

Premature ejaculation is primarily classified into two categories based on etiology and timing: primary premature ejaculation and secondary premature ejaculation. Primary premature ejaculation refers to the inability to control ejaculation time from the first sexual experience, which may be related to physiological or psychological factors, such as issues with testosterone levels and sexual psychological disorders. Secondary premature ejaculation refers to the inability to control ejaculation that occurs after a period of time since the first sexual experience, which may be caused by physiological and psychological reasons, such as marital discord, tension, anxiety, psychological trauma, reproductive system infections, and prostatitis.

The mechanisms behind premature ejaculation are not fully understood, but various potential factors exist, including psychological, physiological, and environmental factors. Psychological factors include anxiety, tension, depression, and lack of sexual experience; physiological factors include prostatitis, genital infections, orchitis, and abnormal hormone levels; environmental factors include lifestyle (such as poor diet, excessive alcohol and tobacco use, and lack of sleep), work environment, and family and marital issues.

The mechanisms of premature ejaculation are numerous and may vary due to differing subjective opinions among doctors. The widely accepted criteria are: (1) ejaculation occurring in less than 1-3 minutes or inability to control ejaculation time; (2) ejaculation time significantly shorter than the time satisfactory to both partners, with premature ejaculation also defined if the female partner cannot reach orgasm in more than half of sexual encounters; (3) experiencing premature ejaculation in almost every sexual encounter for a duration of 6 months or longer; (4) fewer than 10 to 30 thrusts during penile-vaginal intercourse.

Currently, the criteria for diagnosing premature ejaculation primarily rely on the latency time of ejaculation within the vagina. The criteria are highly subjective. The existing standards for diagnosing premature ejaculation have many drawbacks. During a single sexual encounter, the ejaculation time of the male may be influenced by various factors, including the frequency of thrusts, level of sexual arousal, and position, which can lead to different ejaculation times for the same individual under different circumstances. However, there is currently no measuring instrument capable of quantitatively assessing the degree of premature ejaculation by integrating multiple factors, which reduces the accuracy of clinical diagnosis and severely impacts the formulation of treatment methods for premature ejaculation and treatment effectiveness.

### SUMMARY

An objective of embodiments of the present disclosure is to provide a sexual health capacity measurement and analysis system, method, and device, achieving a quantitative assessment of the degree of premature ejaculation by integrating multiple factors, thereby improving diagnostic accuracy.

To achieve the above objective, embodiments of the present disclosure provide the following solutions:

A sexual health capacity measurement and analysis method, including:
acquiring a relative acceleration of waist movements of a male and a female;
acquiring a start time and an end time of sexual activity to obtain a duration;
acquiring a thrust count, a thrust distance, and a thrust frequency of the male, and/or a thrust count, a thrust distance, and a thrust frequency of the female;
obtaining position data based on the relative acceleration of the waist movements of the male and the female; and
inputting the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

Optionally, said acquiring the relative acceleration of the waist movements of the male and the female specifically includes:
obtaining a waist acceleration of the male during movement by using an inertial measurement unit, where the waist acceleration of the male during movement includes: a_{x_m}(t), a_{y_m} (t), and a_{z_m}(t);
obtaining a waist acceleration of the female during movement by using an inertial measurement unit, where the waist acceleration of the female during movement includes: a_{x_w} (t), a_{y_w}(t), and a_{z_w}(t); and
obtaining the relative acceleration of the waist movements of the male and the female based on the waist acceleration of the male during movement and the waist acceleration of the female during movement, where the relative acceleration of the waist movements of the male and the female includes: aₓ(t), a_{y}(t), and a_{z}(t); x, y, and z represent three directions of three-dimensional coordinate axes, a represents acceleration, m represents male, and w represents female;

$${a}_{x} ( t ) = {a}_{x \_ m} ( t ) - {a}_{x \_ w} ( t ) ;$$

$${a}_{y} ( t ) = {a}_{y \_ m} ( t ) - {a}_{y \_ w} ( t ) ;$$

$${a}_{z} ( t ) = {a}_{z \_ m} ( t ) - {a}_{z \_ w} ( t ) .$$

Optionally, said acquiring the thrust count of the male, and/or the thrust count of the female specifically includes:
acquiring a start time tₛₜₐᵣₜ of a thrust, and obtaining an initial velocity;
obtaining an x-axis velocity \vec{Vₓ(t)}, a y-axis velocity \vec{V_{y}(t)}, and a z-axis velocity \vec{V_{z}(t)} at a current time t based on the initial velocity;
obtaining an x-axis displacement \vec{lₓ(t)}, a y-axis displacement \vec{l_{y}(t)}, and a z-axis displacement \vec{l_{z}(t)} based on the start time tₛₜₐᵣₜ, the current time t, the x-axis velocity, \vec{Vₓ(t)}, the y-axis velocity \vec{V_{y}(t)}, and the z-axis velocity \vec{V_{z}(t)};
obtaining an actual displacement \vec{l(t)}, based on the x-axis displacement \vec{lₓ(t)}, the y-axis displacement \vec{l_{y}(t)}, and the z-axis displacement \vec{l_{z}(t)}; and
determining whether the actual displacement \vec{l(t)} is zero, where when the actual displacement becomes zero, it indicates that one cycle of movement is completed, a current time is recorded as t_{end}, and the thrust count is incremented by 1.

Optionally, said acquiring the thrust distance of the male, and/or the thrust distance of the female specifically includes:
acquiring two consecutive sampling times, including a first sampling time tₙ and a second sampling time tₙ₊₁;
obtaining a change in velocity based on the first sampling time tₙ, the second sampling time tₙ₊₁, and an initial velocity at the first sampling time;
obtaining a velocity V_{x_n+1} at the second sampling time based on the initial velocity at the first sampling time and the change in velocity; and
obtaining the thrust distance of the male and/or the thrust distance of the female based on the first sampling time tₙ, the second sampling time tₙ₊₁, the initial velocity at the first sampling time, and the velocity at the second sampling time.

Optionally, said acquiring the thrust frequency of the male and/or the thrust frequency of the female specifically includes:
obtaining the thrust frequency based on the thrust count.

Optionally, said obtaining the position data based on the relative acceleration of the waist movements of the male and the female specifically includes:
converting relative acceleration data of the waist movements of the male and the female into a vector in an object coordinate system; and obtaining the position data based on a relationship between the vector and a gravity vector;
or,
measuring an angular velocity by using a gyroscope, and integrating the angular velocity to obtain an object rotation angle at each time point; fusing the relative acceleration of the waist movements of the male and the female with the rotation angle using a Kalman filter to obtain fused data; and representing the fused data using quaternions to express the position data;
or,
identifying the relative acceleration of the waist movements of the male and the female by using a posture recognition model to obtain the position data.

Optionally, construction of the hierarchical analysis model specifically includes:
establishing a goal layer, a criterion layer, a first sub-criterion layer, a second sub-criterion layer, and a scheme layer, where the goal layer is used to predict a degree of premature ejaculation; the criterion layer is used to calculate the position data; the first sub-criterion layer is used to calculate the thrust count, thrust distance, and thrust frequency of the male, and/or the thrust count, thrust distance, and thrust frequency of the female; the second sub-criterion layer is used to calculate a distribution of the thrust frequency; and the scheme layer outputs sexual health capacity measurement and analysis results for different users;
constructing comparison matrices for the goal layer, the criterion layer, the first sub-criterion layer, the second sub-criterion layer, and the scheme layer, respectively;
performing hierarchical single sorting and a consistency test, as well as hierarchical total sorting and a consistency test on the comparison matrices;
calculating corresponding membership degrees of the comparison matrices using relative membership degrees;
obtaining a generalized weighted distance based on the corresponding membership degrees;
obtaining the sexual health capacity measurement and analysis results for different users based on the generalized weighted distance.

To achieve the above objective, embodiments of the present disclosure also provide the following solutions:

A sexual health capacity measurement and analysis system, including:
a data acquisition module configured to:
acquire a relative acceleration of waist movements of a male and a female;
acquire a start time and an end time of sexual activity to obtain a duration; and
acquire a thrust count, a thrust distance, and a thrust frequency of the male, and/or a thrust count, a thrust distance, and a thrust frequency of the female;
a posture recognition module connected to the data acquisition module and configured to obtain position data based on the relative acceleration of the waist movements of the male and the female; and
a hierarchical analysis module connected to the data acquisition module and the posture recognition module, and configured to input the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

An electronic device is provided, including a memory, a processor, and a computer program that is stored in the memory and executable by the processor. The processor executes the computer program to implement the sexual health capacity measurement and analysis method.

A non-transitory computer-readable storage medium storing a computer program is provided, where the sexual health capacity measurement and analysis method is implemented when the computer program is executed.

In the embodiments of the present disclosure, in order to comprehensively and objectively assess the degree of premature ejaculation occurrence by combining multiple parameters, the duration of the sexual process, thrust frequency, thrust count, relative displacement of the penis within the vagina, and positions used by both parties during sexual activity are monitored. The sexual health capacity measurement and analysis system employs precise sensor devices and reliable analytical methods to measure and record motion data of sexual activity in real time, providing detailed analysis results. Through the sexual health capacity measurement and analysis system, individuals can obtain assessments of their sexual health and receive relevant suggestions to enhance the quality and satisfaction of their sexual life.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can still be derived from these accompanying drawings by those of ordinary skill in the art without creative efforts.

FIG. 1 is a schematic flowchart of a sexual health capacity measurement and analysis method according to an embodiment of the present disclosure;

FIG. 2 is a schematic structural diagram of a sexual health capacity measurement and analysis system according to an embodiment of the present disclosure;

FIG. 3 is a schematic diagram of a hierarchical analysis module according to an embodiment of the present disclosure;

FIG. 4 is a schematic diagram of a start interface according to an embodiment of the present disclosure;

FIG. 5 is a schematic diagram of a “Start Test” interface according to an embodiment of the present disclosure;

FIG. 6 is a schematic diagram of an “End Test” interface according to an embodiment of the present disclosure;

FIG. 7 is a schematic diagram of a test result according to an embodiment of the present disclosure;

FIG. 8 is a schematic diagram of test result analysis according to an embodiment of the present disclosure;

FIG. 9 is a schematic diagram of the test end according to an embodiment of the present disclosure;

FIG. 10 is a detailed schematic diagram of a sexual health capacity measurement and analysis system according to an embodiment of the present disclosure;

FIG. 11 is a schematic diagram of modules of a sexual health capacity measurement and analysis system according to an embodiment of the present disclosure;

FIGS. 12A-12B are schematic diagrams of male and female testing according to an embodiment of the present disclosure; and

FIG. 13 is a schematic diagram of interconnection of a sexual health capacity measurement and analysis system according to an embodiment of the present disclosure.

### DESCRIPTION OF REFERENCE NUMERALS

101: data acquisition module; 102: posture recognition module; 103: hierarchical analysis module.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a sexual health capacity measurement and analysis system, method, and device, to address the issues of incomplete factors in the current assessment of premature ejaculation and low diagnostic accuracy.

The embodiments of the present disclosure include three main objectives:
1. overcoming the drawbacks of traditional premature ejaculation diagnosis, which primarily relies on the latency time of ejaculation within the vagina as the basis for judgment;
2. monitoring the duration of the sexual process, thrust frequency, thrust count, relative displacement of the penis within the vagina, and positions used by both parties during sexual activity, to objectively and comprehensively assess the degree of premature ejaculation occurrence by integrating multiple factors;
3. developing a multi-parameter, multi-level evaluation method for premature ejaculation, which includes two interchangeable, identical subsystems: one worn by females and the other by males. Generally, the subsystem worn by males is considered the main subsystem, while the one worn by females is regarded as the auxiliary subsystem. The descriptions provided below will follow this convention.

In order to make the above objective, features and advantages of the present disclosure clearer and more comprehensible, the present disclosure will be further described in detail below in combination with accompanying drawings and particular implementation modes.

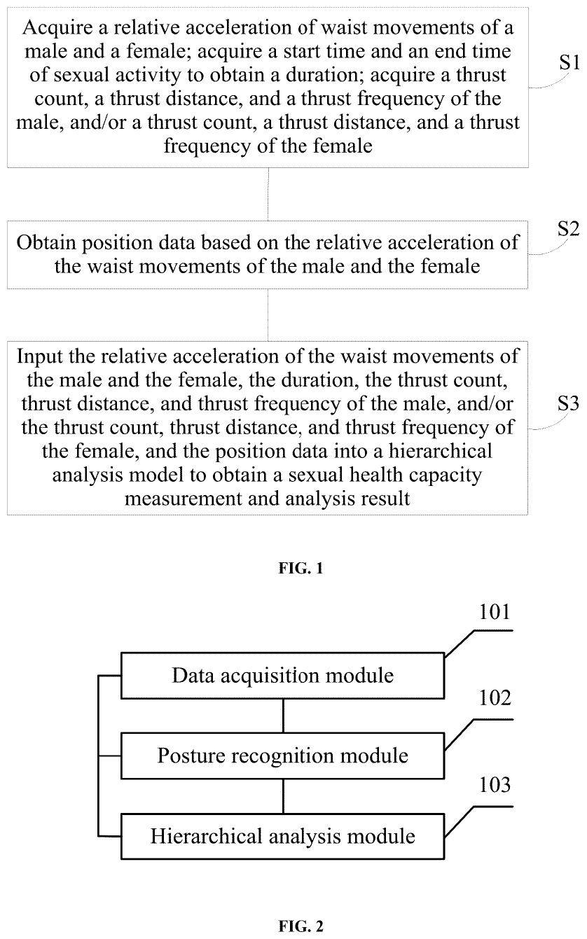

FIG. 1 shows an exemplary flow of a sexual health capacity measurement and analysis method. The following provides a detailed introduction to each step.

S1: Acquire a relative acceleration of waist movements of a male and a female;
acquire a start time and an end time of sexual activity to obtain a duration.

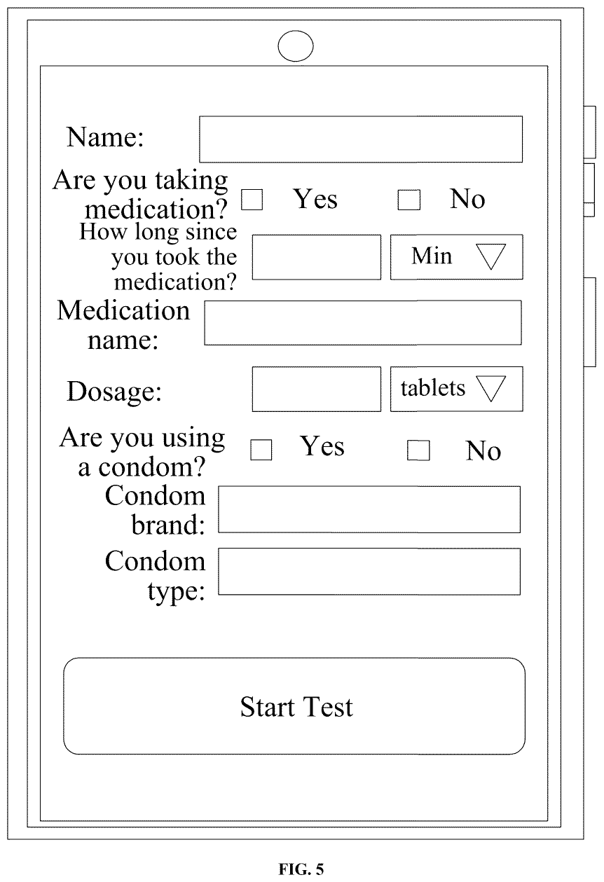

In one example, as shown in FIG. 5, the start time is the moment the “Start Test” button is clicked.

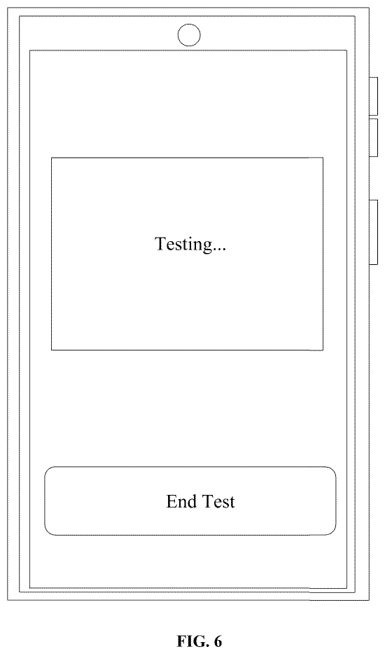

As shown in FIG. 6, the duration is the time from the moment the “Start Test” button is clicked to the moment the “End Test” button is clicked.

Acquire a thrust count, a thrust distance, and a thrust frequency of the male, and/or a thrust count, a thrust distance, and a thrust frequency of the female.

Said acquiring the relative acceleration of the waist movements of the male and the female specifically includes:

Step S11: Obtain a waist acceleration of the male during movement by using an inertial measurement unit, where the waist acceleration of the male during movement includes: a_{x_m}(t), a_{y_m}(t), and a_{z_m}(t).

Step S12: Obtain a waist acceleration of the female during movement by using an inertial measurement unit, where the waist acceleration of the female during movement includes: a_{x_m}(t), a_{y_w}(t), and a_{z_w}(t).

S13: Obtain the relative acceleration of the waist movements of the male and the female based on the waist acceleration of the male during movement and the waist acceleration of the female during movement, where the relative acceleration of the waist movements of the male and the female includes: aₓ(t), a_{y}(t), and a_{z}(t); x, y, and z represent three directions of three-dimensional coordinate axes, a represents acceleration, m represents male, and w represents female;

$${a}_{x} ( t ) = {a}_{x \_ m} ( t ) - {a}_{x \_ w} ( t ) ;$$

$${a}_{y} ( t ) = {a}_{y \_ m} ( t ) - {a}_{y \_ w} ( t ) ;$$

$$and$$

$${a}_{z} ( t ) = {a}_{z \_ m} ( t ) - {a}_{z \_ w} ( t ) .$$

In one example, a sensor integration system capable of collecting data needs to be powered on in a stationary state. The main subsystem obtains the waist acceleration of the male during movement through the inertial measurement unit, with accelerations along the three axes being: a_{x_m} (t), a_{y_m} (t), and a_{z_m} (t); the auxiliary subsystem obtains the waist acceleration of the female, during movement through the inertial measurement unit, with accelerations along the three axes being: a_{x_w}(t), a_{y_w}(t), and a_{z_w}(t).

The sensor integration system is placed at the waist, close to the external genitalia of both the male and female, allowing waist movement data during intercourse to be approximated as movement data of the external genitalia. The following data collection principles analyze the relative movements of the male and female.

Optionally, said acquiring the thrust count of the male, and/or the thrust count of the female specifically includes:

Steps S14: Acquire a start time tₛₜₐᵣₜ chart of a thrust, and obtain an initial velocity.

Step S15: Obtain an x-axis velocity \vec{Vₓ(t)}, a y-axis velocity \vec{V_{y}(t)}, and a z-axis velocity \vec{V_{z}(t)} at a current time based on the initial velocity.

Step S16: Obtain an x-axis displacement \vec{lₓ(t)}, a y-axis displacement \vec{l_{y}(t)}, and a z-axis displacement \vec{L_{Z}(T)} based on the start time tₛₜₐᵣₜ, the current time t, the x-axis velocity, \vec{Vₓ(t)}, the y-axis velocity \vec{V_{y}(t)}, and the z-axis velocity \vec{V_{z}(t)}.

Step S17: Obtain an actual displacement \vec{l(t)} based on the x-axis displacement \vec{lₓ(t)}, the y-axis displacement \vec{l_{y}(t)}, and the z-axis displacement \vec{l_{z}(t)}.

Step S18: Determine whether the actual displacement \vec{l(t)} is zero, where when the actual displacement becomes zero, it indicates that one cycle of movement is completed, a current time is recorded as t_{end}, and the thrust count is incremented by 1.

In one example, the start time of the thrust is recorded as tₛₜₐᵣₜ, with the initial velocity being:

$$\vec{{V}_{x \_ start}} = {\int }_{0}^{{t}_{start}} {a}_{x} ( t ) dt .$$

The current time is t, at which point the x-axis velocity is:

$$\vec{{V}_{x} ( t )} = \vec{{V}_{x \_ start}} + {\int }_{{t}_{start}}^{t} {a}_{x} ( t ) dt .$$

Similarly, the y-axis velocity \vec{V_{y}(t)} and z-axis velocity \vec{V_{z}(t)} can be calculated, and thus the x-axis displacement at this moment is:

$$\vec{{l}_{x} ( t )} = {\int }_{{t}_{start}}^{t} \vec{{V}_{x} ( t )} dt .$$

Likewise, the y-axis displacement \vec{l_{y}(t)} and z-axis displacement \vec{l_{z}(t)} can be calculated. Since the velocity and displacement are both vectors, the actual displacement is a vector sum of the three-axis displacements:

$$\vec{l ( t )} = \vec{{l}_{x} ( t )} + \vec{{l}_{y} ( t )} + \vec{{l}_{z} ( t )} .$$

When the actual displacement becomes zero, it indicates that the user has completed one cycle of movement, and the time at this moment is recorded as t_{end}, and the thrust count is incremented by one. An array array_log is set up, the start time tₛₜₐᵣₜ of this thrust is added to array_log, and t_{end} is recorded as new tₛₜₐᵣₜ, starting the next round of thrust count monitoring.

Said acquiring the thrust distance of the male, and/or the thrust distance of the female specifically includes:

Step S19: Acquire two consecutive sampling times, including a first sampling time tₙ and a second sampling time tₙ₊₁.

Step S110: Obtain a change in velocity based on the first sampling time tₙ, the second sampling time tₙ₊₁, and an initial velocity at the first sampling time.

Step S111: Obtain a velocity V_{x_n+1} at the second sampling time based on the initial velocity at the first sampling time and the change in velocity.

Step S112: Obtain the thrust distance of the male and/or the thrust distance of the female based on the first sampling time tₙ, the second sampling time tₙ₊₁, the initial velocity at the first sampling time, and the velocity at the second sampling time.

In one example, the two consecutive sampling times are tₙ and tₙ₊₁. Since the time interval between two sampling points is short, the acceleration can be approximated as a linear change. The initial velocity is V_{x_n}, which is calculated in the same manner as above, thus the change in velocity along the x-axis from tₙ to tₙ₊₁ is:

$$Δ {V}_{x} = \frac{( {a}_{x \_ n} - {a}_{x \_ n + 1} ) ( {t}_{n + 1} - {t}_{n} )}{2} .$$

At the sampling time tₙ₊₁, the x-axis velocity is V_{x_n+1}:

$${V}_{x \_ n + 1} = {V}_{x \_ n} + Δ {V}_{x} .$$

where a_{x_n} and a_{x_n+1} represent x-axis acceleration values at moments tₙ and tₙ₊₁, respectively. Similarly, ΔV_{y} and ΔV₂ can be obtained, thus the change in distance along the x-axis from tₙ to tₙ₊₁ is:

$$Δ {S}_{x} = \frac{( {V}_{x \_ n} - {V}_{x \_ n + 1} ) ( {t}_{n + 1} - {t}_{n} )}{2} .$$

Likewise, ΔS_{y} and ΔS_{z} can be obtained, thus an actual movement distance from tₙ to tₙ₊₁ is:

$${S}_{n} = \sqrt{Δ {S}_{x}^{2} + Δ {S}_{y}^{2} + {S}_{z}^{2}} .$$

A total movement distance, that is, a total thrust distance, is:

$${S}_{sum} = {\sum }_{t = 1}^{n} {S}_{t} .$$

where n is the total number of monitored sampling points.

Said acquiring the thrust frequency of the male and/or the thrust frequency of the female specifically includes:

Step S113: Obtain the thrust frequency based on the thrust count.

In one example, the frequency is inversely related to the time interval. By using the recorded start time of each thrust during the monitoring of the thrust count, the time interval between adjacent thrusts is calculated to reflect changes in frequency. Quick thrusts have shorter time intervals, while slow thrusts have longer time intervals. The number of time intervals within different ranges during the monitoring process is counted, and any two adjacent moments tₘ and tₘ₊₁ recorded in the backend array array_log are extracted to calculate the time interval lₘ:

$${I}_{m} = {t}_{m + 1} - {t}_{m} .$$

The time intervals are categorized as follows: shorter than 0.3 seconds, 0.3-0.4 seconds, 0.4-0.5 seconds, 0.5-0.6 seconds, 0.6-0.7 seconds, 0.7-0.8 seconds, 0.8-0.9 seconds, 0.9-1 second, and greater than 1 second for statistical analysis, and a statistical chart is drawn.

Step S2 of obtaining the position data based on the relative acceleration of the waist movements of the male and the female specifically includes:

Step S21: Convert relative acceleration data of the waist movements of the male and the female into a vector in an object coordinate system; and obtain the position data based on a relationship between the vector and a gravity vector.

Or,

S22: Measure an angular velocity by using a gyroscope, and integrate the angular velocity to obtain an object rotation angle at each time point; fuse the relative acceleration of the waist movements of the male and the female with the rotation angle using a Kalman filter to obtain fused data; and represent the fused data using quaternions to express the position data.

Or,

Step S23: Identify the relative acceleration of the waist movements of the male and the female by using a posture recognition model to obtain the position data.

In one example, the accelerometer in the sensor integration system can measure the acceleration of an object. By converting the measurement data of the accelerometer into a vector in the object coordinate system, and based on the relationship between the vector and the gravity vector, the posture of the object can be determined.

In another example, the gyroscope in the sensor integration system can measure the rotational rate (i.e., angular velocity) of the object around its three axes. By integrating the measurement data of the gyroscope, the rotation angle of the object at each time point can be obtained. The data from the accelerometer and gyroscope are fused using a Kalman filter; based on the fused data, quaternions are used to express the posture.

In another example, artificial intelligence methods are used for training: identifying the current position based on the posture data collected by the sensor integration system, and labeling all data with positions.

Step S3: Input the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

Construction of the hierarchical analysis model specifically includes:

Step S31: Establish a goal layer, a criterion layer, a first sub-criterion layer, a second sub-criterion layer, and a scheme layer, where the goal layer is used to predict a degree of premature ejaculation; the criterion layer is used to calculate the position data; the first sub-criterion layer is used to calculate the thrust count, thrust distance, and thrust frequency of the male, and/or the thrust count, thrust distance, and thrust frequency of the female; the second sub-criterion layer is used to calculate a distribution of the thrust frequency; and the scheme layer outputs sexual health capacity measurement and analysis results for different users.

Step S32: Construct comparison matrices for the goal layer, the criterion layer, the first sub-criterion layer, the second sub-criterion layer, and the scheme layer, respectively.

Step S33: Perform hierarchical single sorting and a consistency test, as well as hierarchical total sorting and a consistency test on the comparison matrices.

Step S34: Calculate corresponding membership degrees of the comparison matrices using relative membership degrees.

Step S35: Obtain a generalized weighted distance based on the corresponding membership degrees.

Step S36: Obtain the sexual health capacity measurement and analysis results for different users based on the generalized weighted distance.

In one example, data collection is conducted with 1,000 healthy male volunteers, with each volunteer providing 10 sets of data for each position. Data is collected for three positions: “doggy style,” “cowgirl,” and “waist-high position,” totaling 30,000 sets of data. The average data in the monitoring of the duration, thrust count, thrust distance, and thrust frequency is calculated for each position, denoted as: T_{s_pos}, N_{s_pos}, S_{s_pos}, and F_{s_pos}[m], where pos takes values of “doggy style,” “cowgirl,” and “waist-high position,” and F_{s_pos}[m] indicates the number of each time interval in the thrust frequency monitoring. The average data is defined as standard data, that is, a standard for the score of 100.

After the collection of user data, the analytic hierarchy process (AHP) is used to decompose the evaluation indicators into multiple components. Next, all components are grouped according to specific relationships to obtain a corresponding hierarchical structure. The specific steps are as follows:

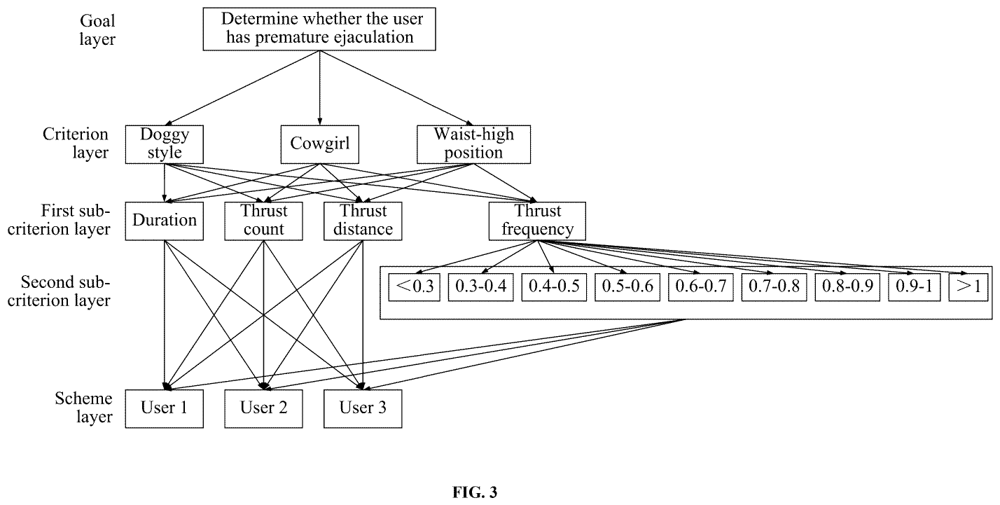

As shown in FIG. 3, the goal layer, criterion layer, first sub-criterion layer, second sub-criterion layer, and scheme layer are established. The goal layer is to assess the degree of premature ejaculation in patients. The attributes of the criterion layer are the positions used, the attributes of the first sub-criterion layer are duration, thrust count, thrust distance, and thrust frequency, while the second sub-criterion layer pertains to the distribution of thrust frequency, and the scheme layer represents different users.

The method proposed by Saaty and others is typically used for determining weights of each attribute and the components of each attribute. For a given attribute set, pairwise comparisons of the elements within the set are made, with their importance represented by aᵢⱼ. The values of aᵢⱼ are quantified using the method in Table 1.

TABLE 1

| | Quantified |
|---|---|
| Judgment Condition | Value |
| Element i is equally important as element j | 1 |
| Element i is slightly more important than element j | 3 |
| Element i is strongly more important than element j | 5 |
| Element i is very strongly more important than element j | 7 |
| Element i is extremely more important than element j | 9 |
| Intermediate degrees of the above judgment conditions | 2, 4, 6, 8 |

A hierarchical judgment matrix A=(aᵢⱼ)_{n×n} is constructed, where n is the number of elements in the attribute set, aᵢⱼ=1/aⱼᵢ, and aᵢₜ=1.

In the present disclosure, a hierarchical judgment matrix A for the criterion layer against the goal layer is constructed as follows:

$$A = [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] .$$

Hierarchical judgment matrices B₁, B₂, and B₃ for the first sub-criterion layer against the criterion layer are constructed:

$${B}_{x} = [ \begin{matrix}{b}_{x \_ 11} & {b}_{x \_ 12} & {b}_{x \_ 13} & {b}_{x \_ 14} \\ {b}_{x \_ 21} & {b}_{x \_ 22} & {b}_{x \_ 23} & {b}_{x \_ 24} \\ {b}_{x \_ 31} & {b}_{x \_ 32} & {b}_{x \_ 33} & {b}_{x \_ 34} \\ {b}_{x \_ 41} & {b}_{x \_ 42} & {b}_{x \_ 43} & {b}_{x \_ 44}\end{matrix} ] .$$

where x takes values of 1, 2, and 3, corresponding to judgment matrices for standing, supine, and sitting positions of the first sub-criterion layer.

A hierarchical judgment matrix C for the second sub-criterion layer against the sub-criterion layer 1 is constructed:

$$C = [ \begin{matrix}{c}_{11} & … & {c}_{19} \\ ⋮ & ⋱ & ⋮ \\ {c}_{91} & … & {c}_{99}\end{matrix} ] .$$

In one example, an eigenvalue λ of the matrix A is calculated, and a corresponding normalized eigenvector W is found, where the eigenvector Wₘₐₓ corresponds to the largest eigenvalue λₘₐₓ represents a ranking result for the current layer, indicating importance ranking weights of the elements in the current layer relative to a specific factor in the previous layer.

To ensure the consistency of the judgment matrix, the matrix needs to satisfy the following condition: aᵢⱼ·aⱼₖ=aᵢₖ, that is, the matrix A is a positive reciprocal consistent matrix. It is determined whether A can be used for analytical decision making of the target by judging its consistency. Assuming that the matrix A is an n-order matrix, a greater difference between λₘₐₓ and n indicates higher inconsistency of the matrix. The matrix is consistent if and only if λₘₐₓ equals n. Therefore, the difference between the two can be used to measure the consistency of the matrix. A consistency index CI is defined as follows:

$$CI = \frac{{λ}_{max} - n}{n - 1} .$$

When CI is zero, the matrix is completely consistent; a larger value of CI indicates higher inconsistency of the matrix. When the number of matrices is not one, to measure the size of multiple CI, a random consistency index RI is defined as follows:

$$RI = \frac{{CI}_{1} + {CI}_{2} + … + {CI}_{n}}{n} .$$

RI is related to n, and the relationship is as shown in Table 2.

TABLE 2

| n | 1 | 2 | 3 | 4 | 5 | 6 | 7 | 8 | 9 |
|---|---|---|---|---|---|---|---|---|---|
| RI | 0.00 | 0.00 | 0.85 | 0.90 | 1.12 | 1.24 | 1.32 | 1.41 | 1.45 |

To determine whether the consistency of the matrix is satisfactory, a verification coefficient CR is defined as follows:

$$CR = \frac{CI}{RI} .$$

When CR<0.1, the matrix is accepted; otherwise, the matrix is not accepted.

In one example, the hierarchical total ranking is the ranking of the importance of intermediate layer attributes regarding the goal layer, and the consistency is checked.

In another example, the minimum attribute value r_{i,min} corresponds to a relative membership degree of 0, while the maximum attribute value r_{i,max} corresponds to a relative membership degree of 1. A relative membership degree matrix R is constructed for U using a relative membership degree function:

$${r}_{ij} = \{ \begin{matrix}0 & {x}_{ij} \leq {r}_{i , min} \\ \frac{{x}_{ij} - {r}_{i , min}}{{r}_{i , max} - {r}_{i , min}} & {r}_{i , min} \leq {x}_{ij} \leq {r}_{i , max} \\ 1 & {r}_{i , max} < {x}_{ij}\end{matrix} .$$

In another example, the relative membership degrees of 1 and 0 correspond to the superiority and inferiority of the samples, respectively. The generalized weighted distances for the samples regarding superiority and inferiority are calculated as follows:

$${d}_{j 1} = \{ {\sum }_{i = 1}^{m} {[ {w}_{i} ( 1 - {r}_{ij} ) ]}^{2} \} , j = 1 , 2 , … , n ; and$$

$${d}_{j 0} = \{ {\sum }_{i = 1}^{m} {[ {w}_{i} ( {r}_{ij} - 0 ) ]}^{2} \} , j = 1 , 2 , … , n .$$

The generalized weighted distances are normalized to construct a relative membership degree function for superiority:

$${S}_{j} = \frac{{\sum }_{i = 1}^{m} {( {w}_{i} {r}_{ij} )}^{2}}{{\sum }_{i = 1}^{m} {( {w}_{i} {r}_{ij} )}^{2} + {{\sum }_{i = 1}^{m} [ {w}_{i} ( 1 - {r}_{ij} ) ]}^{2}} .$$

In another example, a quantitative value for evaluating this fuzzy subset is:

$$N = \frac{1}{n} {\sum }_{j = 1}^{n} {S}_{j} .$$

Finally, a normalized score for this test is obtained, where the value falls in a range of 0 to 1. This value is multiplied by 100 to give the score for the patient in this test. A patient having a score below 60 is diagnosed with premature ejaculation, and this score also represents the sexual endurance and ejaculation control ability of the user.

### Embodiment 1

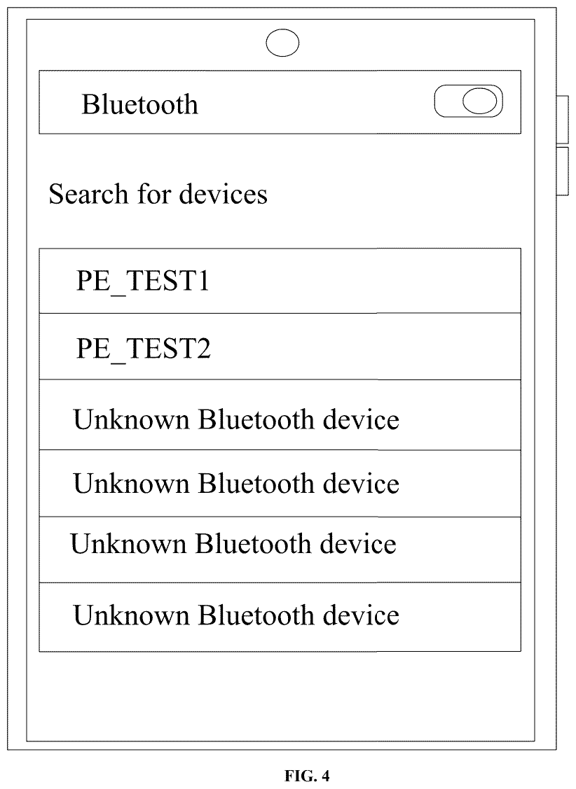

Step 1: Referring to FIG. 4, open a testing device and prepare for the test: Open the WeChat applet or mobile application “Self-Testing Sexual Health Capacity Measurement and Analysis System—General Version.” Click to enter the test preparation page to fill in the following information according to the prompts: “Name,” “Are you taking medication?” “How long since you took the medication?” “Medication name,” “Medication amount,” “Are you using a condom?” “Condom brand,” and “Condom type.” The units for “How long since you took the medication?” and “Medication amount” can be selected from a dropdown menu. The unit for “How long since you took the medication?” can be either “hours” or “minutes,” and the unit for “Medication amount” can be “tablets,” “ml,” or “mg.”

Step 2: Referring to FIG. 5, install the device: In a stationary state, click the “Start Test” button to enable the phone to enter the monitoring process, displaying “Testing . . . ”. After starting the test, male and female users will each secure the belt around the waist and attach the mobile device to a mobile device fixing box on the belt.

Step 3: Referring to FIG. 6, monitoring process: After the above steps, sexual intercourse can begin. During the monitoring process, the phone screen will display “Testing . . . ” to indicate the current testing process, while the backend will record test data in real time. There is an “End Test” button at the bottom.

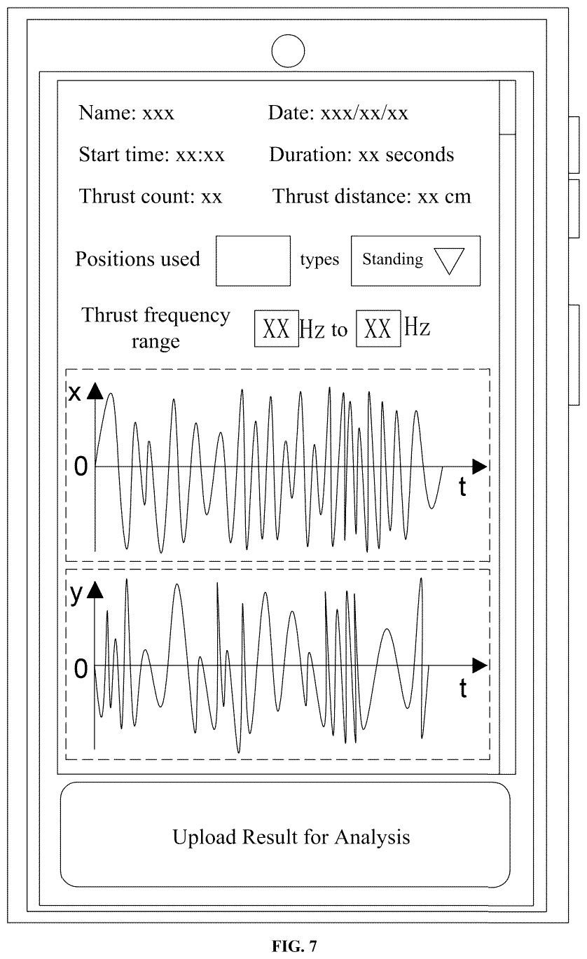

Step 4: Referring to FIG. 7, end the test: After the test is completed, click the “End Test” button as soon as possible. After the test ends, the page will display “Name,” “Date,” “Start Time,” “Duration,” “Thrust Count,” “Positions Used,” “Thrust Frequency Range,” and waveforms of x, y, z-axis acceleration magnitudes over time, as well as a map of frequency range distribution. The “Positions Used” has a dropdown option that can display the positions identified during this test, including: “Doggy Style,” “Cowgirl,” and “Waist-High Position,” etc. Switching the dropdown option will trigger a data switch, and this page will only display all data for the current position.

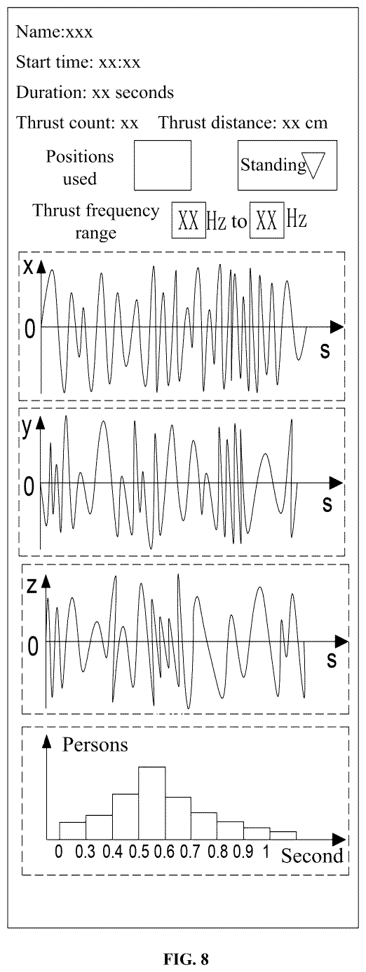

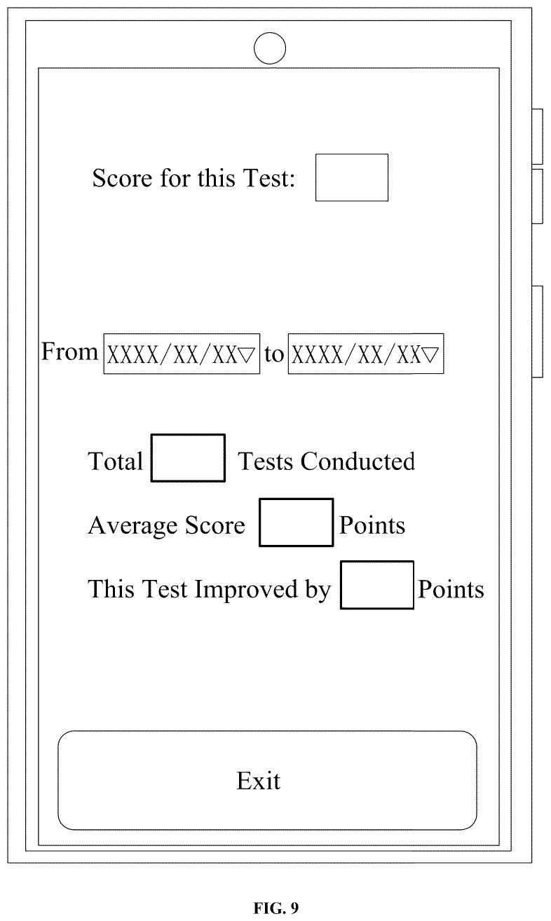

Step 5: Referring to FIG. 8 and FIG. 9, upload analysis results: Click “Upload Result for Analysis” to upload the test data to the server for scoring analysis. The page will display “Score for this Test,” where a time period can be selected for data analysis, and the page will show “Total X Tests Conducted,” “Average Score X Points,” and “This Test Improved by X Points.”

Step 6: Exit the test: Click the “Exit” button to leave the “Sexual Health Capacity Measurement and Analysis System” applet.

In summary, in the embodiments of the present disclosure, in order to comprehensively and objectively assess the degree of premature ejaculation occurrence by combining multiple parameters, the duration of the sexual process, thrust frequency, thrust count, relative displacement of the penis within the vagina, and positions used by both parties during sexual activity are monitored. The sexual health capacity measurement and analysis system employs precise sensor devices and reliable analytical methods to measure and record motion data of sexual activity in real time, providing detailed analysis results. Through the sexual health capacity measurement and analysis system, individuals can obtain assessments of their sexual health and receive relevant suggestions to enhance the quality and satisfaction of their sexual life.

To achieve the above objective, embodiments of the present disclosure also provide the following solutions:

As shown in FIG. 2, a sexual health capacity measurement and analysis system is provided, including a data acquisition module 101, a posture recognition module 102, and a hierarchical analysis module 103.

The data acquisition module 101 (namely, a sensor integration system) is configured to:
acquire a relative acceleration of waist movements of a male and a female;
acquire a start time and an end time of sexual activity to obtain a duration; and
acquire a thrust count, a thrust distance, and a thrust frequency of the male, and/or a thrust count, a thrust distance, and a thrust frequency of the female.

The posture recognition module 102 is connected to the data acquisition module 101 and is configured to obtain position data based on the relative acceleration of the waist movements of the male and the female.

The hierarchical analysis module 103 is connected to the data acquisition module 101 and the posture recognition module 102, and is configured to input the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

### Embodiment 2

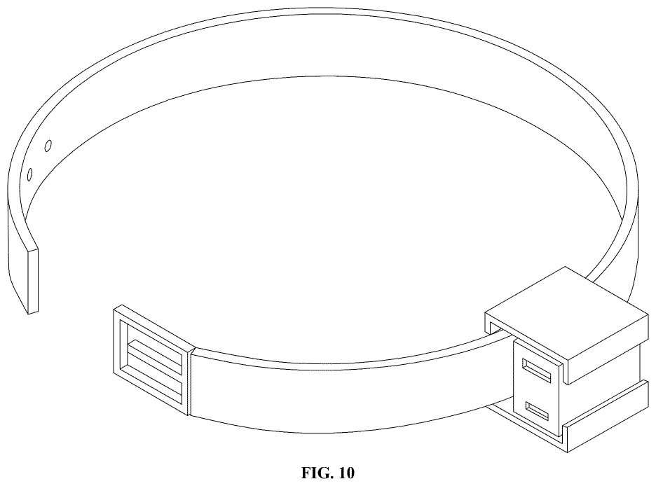

Referring to FIG. 10, the sexual health capacity measurement and analysis system of the present disclosure includes two components: motion measurement equipment and visualization interactive software. The motion measurement equipment is divided into two types: dedicated measurement equipment and general measurement equipment. One type uses inertial measurement devices as dedicated measurement equipment, achieving the advantages of accurate measurement, personalized customization, and applicability for clinical diagnosis or scientific research data acquisition. The other type uses mobile phones or other mobile devices equipped with gyroscopes and accelerometers as general measurement equipment, which is simple to use and does not require the purchase of additional devices, making it suitable for home self-testing. The visualization interactive software is installed on the mobile device and incorporates the multi-parameter evaluation method for premature ejaculation designed in the present disclosure, capable of displaying the measurement process and results for human-computer interaction, processing backend data, and uploading measurement results. The dedicated equipment in the sexual health capacity measurement and analysis system of the present disclosure includes two completely identical, interchangeable subsystems. Each subsystem includes a belt, a sensor integration system, a fixing box for the sensor integration system, and a mobile device. Referring to FIG. 11, the belt is equipped with a fixing box for the sensor integration system, which consists of an upper fixing clip and a lower fixing clip to secure the sensor integration system. The sensor integration system integrates an inertial measurement unit, a controller, a memory, a Bluetooth control chip, a Bluetooth antenna, a power module, and other electronic components into one device, and is protected by a casing that includes a power switch and a TYPE-C charging port. The visualization interactive software interacts with the sensor integration system via Bluetooth, processes and evaluates the acquired data using the multi-parameter evaluation method for premature ejaculation, and displays processing results on a corresponding display interface of the visualization interactive software. In the present disclosure, positions are used as labels, and only data for the current position is displayed each time. Switching the position label will switch the data.

The design of the two types of motion measurement equipment in the sexual health capacity measurement and analysis system according to the embodiments of the present disclosure includes dedicated measurement equipment for clinical diagnosis or scientific research and general measurement equipment for home self-testing.

The two types of motion measurement equipment in the sexual health capacity measurement and analysis system according to the embodiments of the present disclosure have different operational steps.

The design of the dedicated measurement equipment in the sexual health capacity measurement and analysis system according to the embodiments of the present disclosure includes a belt, a sensor integration system, a fixing box for the sensor integration system, and a mobile device.

Referring to FIG. 13, the design of the general measurement equipment in the sexual health capacity measurement and analysis system of the present disclosure includes a belt, a mobile device equipped with an accelerometer and a gyroscope, and a fixing box for the mobile device, where the mobile device includes phones, smartwatches, etc.

The design of the sensor integration system in the dedicated measurement equipment of the present disclosure includes an inertial measurement unit, a controller, a memory, a Bluetooth control chip, a Bluetooth antenna, a power module, and other electronic components.

The method of data collection using the inertial measurement unit in the embodiments of the present disclosure is as follows:

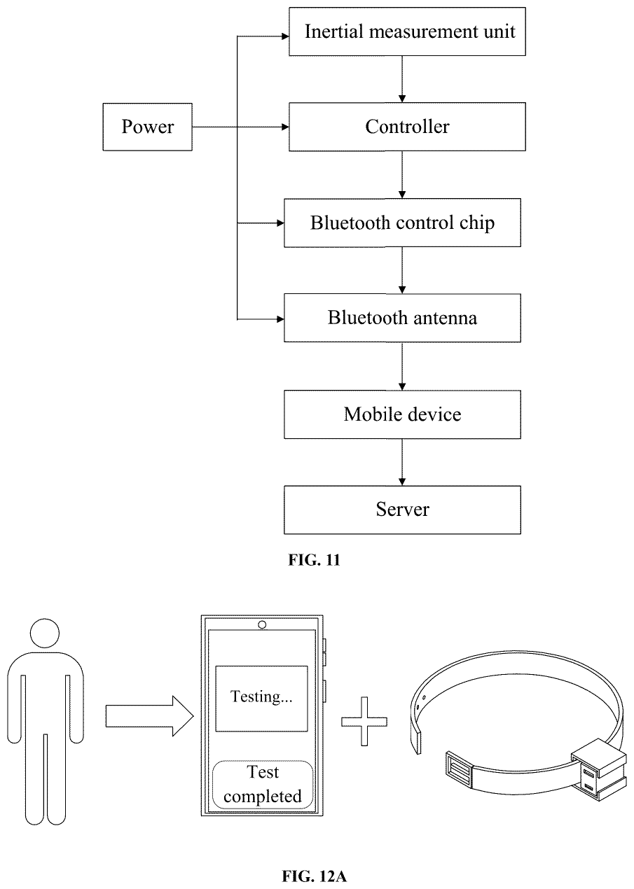

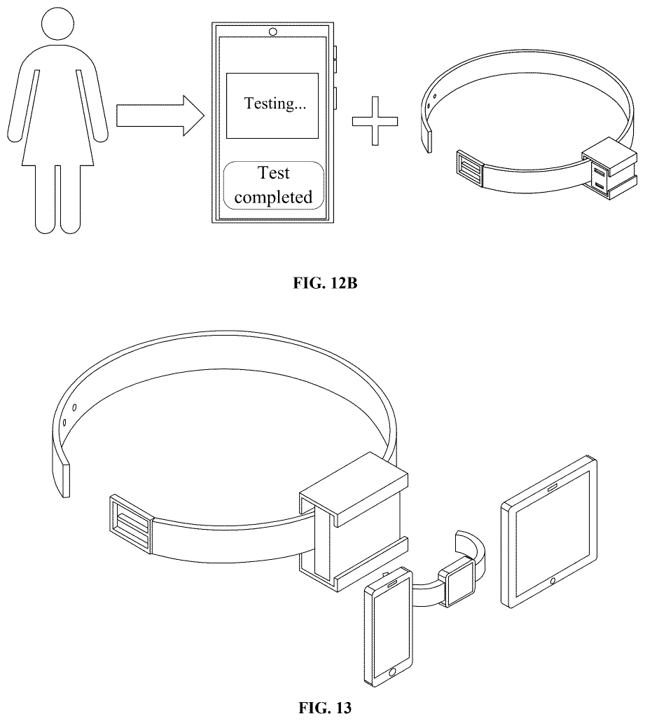

Referring to FIGS. 12A-12B, in the embodiments of the present disclosure, two identical subsystems are used to collect data from the male and female separately to obtain data on their relative movements.

The method for collecting data on the degree of premature ejaculation occurrence defined in the embodiments of the present disclosure includes collecting a thrust count and thrust distance, thrust frequency monitoring, position monitoring, and finally providing a quantified score to define the degree of premature ejaculation quantitatively.

The monitoring method for the thrust count in the embodiments of the present disclosure includes using the accelerometer to capture moments when displacement is zero, and recording each occurrence of zero displacement as one thrust.

The monitoring method for thrust distance in the embodiments of the present disclosure includes using the accelerometer to obtain movement distances for each sampling interval, and summing the movement distances of all sampling intervals to obtain a total thrust distance.

The monitoring method for thrust frequency in the embodiments of the present disclosure includes calculates time intervals for each thrust based on the moments of zero displacement obtained from the thrust count monitoring, where the time intervals reflect the magnitude of frequency, and statistically analyzing the data for different time intervals to derive the frequency distribution.

The method for position monitoring in the embodiments of the present disclosure includes using the accelerometer and gyroscope to calculate a posture angle and employing an artificial intelligence method to identify the current position.

In the embodiments of the present disclosure, the acquired data is processed using the analytic hierarchy process and fuzzy decision-making methods to calculate a quantified value for the degree of premature ejaculation occurrence.

The embodiments of the present disclosure provide a scoring method for sexual behavior in the software, allowing users to intuitively understand the degree of their premature ejaculation.

The design of the fixing box for the sensor integration system in the embodiments of the present disclosure includes two fixing clips at the top and bottom, which can use springs, bolts, limiters, or any other means of fixation.

In the embodiments of the present disclosure, the sexual health capacity measurement and analysis system is designed as two subsystems, with the main subsystem for male use and the auxiliary subsystem for female use, allowing the collection of relative motion information from both parties.

The sexual health capacity measurement and analysis system designed in the embodiments of the present disclosure diagnose the degree of premature ejaculation occurrence by collecting multiple parameters.

In the embodiments of the present disclosure, a mobile device is used as a host computer to receive and process signals, where the mobile device includes a phone, a tablet, a smartwatch, and the like.

In the embodiments of the present disclosure, a Bluetooth module is used as a data transmission means.

Further, the present disclosure further provides an electronic device. The electronic device may include: a processor, a communication interface, a memory and a communication bus. The processor, the communication interface and the memory communicate with one another by means of the communication bus. The processor can invoke the computer program in the memory and execute the computer program to implement the sexual health capacity measurement and analysis method.

In addition, the computer program in the above memory may be stored in a computer-readable storage medium when the computer program is implemented in a form of a software function unit and is sold or used as an independent product. On the basis of such understanding, the technical solutions of the present disclosure essentially or the part contributing to the prior art may be embodied in a form of a software product. The computer software product is stored in a storage medium, and includes several instructions for enabling a computer device (which may be a personal computer, a server, a network device, etc.) to execute all or some steps of the methods described in the embodiments of the present disclosure. The above storage medium includes any medium that may store program codes, such as a USB flash drive, a removable hard disk, a read-only memory, a random access memory, a magnetic disk and an optical disc.

The present disclosure further provides a non-transitory computer storage medium storing a computer program, where the sexual health capacity measurement and analysis method is implemented when the computer program is executed.

Each embodiment in the description is described in a progressive mode; each embodiment focuses on differences from other embodiments, and references can be made to each other for the same and similar parts between embodiments. Since the system disclosed in an embodiment corresponds to the method disclosed in an embodiment, the description is relatively simple, and for related contents, references can be made to the description of the method.

Specific examples are used herein for illustration of principles and embodiments of the present disclosure. The descriptions of the above embodiments are merely used for assisting in understanding the methods and core ideas of the embodiments of the present disclosure. In addition, those of ordinary skill in the art can make various modifications in terms of the particular implementations and the scope of application in accordance with the ideas of the present disclosure. In conclusion, the content of the description shall not be construed as limitations to the embodiments of the present disclosure.

1. A sexual health capacity measurement and analysis method, comprising:
acquiring a relative acceleration of waist movements of a male and a female;
acquiring a start time and an end time of sexual activity to obtain a duration;
acquiring a thrust count, a thrust distance, and a thrust frequency of the male, and/or a thrust count, a thrust distance, and a thrust frequency of the female;
obtaining position data based on the relative acceleration of the waist movements of the male and the female; and
inputting the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

2. The sexual health capacity measurement and analysis method according to claim 1, wherein said acquiring the relative acceleration of the waist movements of the male and the female specifically comprises:
obtaining a waist acceleration of the male during movement by using an inertial measurement unit, wherein the waist acceleration of the male during movement comprises: a_{x_m}(t), a_{y_m}(t), and a_{z_m}(t);
obtaining a waist acceleration of the female during movement by using an inertial measurement unit, wherein the waist acceleration of the female during movement comprises: a_{x_w}(t), a_{y_w}(t), and a_{z_w}(t); and
obtaining the relative acceleration of the waist movements of the male and the female based on the waist acceleration of the male during movement and the waist acceleration of the female during movement, wherein the relative acceleration of the waist movements of the male and the female comprises: aₓ(t), a_{y}(t), and a_{z}(t); x, y, and z represent three directions of three-dimensional coordinate axes, a represents acceleration, m represents male, and w represents female;
$${a}_{x} ( t ) = {a}_{x \_ m} ( t ) - {a}_{x \_ w} ( t ) ;$$
$${a}_{y} ( t ) = {a}_{y \_ m} ( t ) - {a}_{y \_ w} ( t ) ;$$
$${a}_{z} ( t ) = {a}_{z \_ m} ( t ) - {a}_{z \_ w} ( t ) .$$

3. The sexual health capacity measurement and analysis method according to claim 1, wherein said acquiring the thrust count of the male, and/or the thrust count of the female specifically comprises:
acquiring a start time tₛₜₐᵣₜ of a thrust, and obtaining an initial velocity;
obtaining an x-axis velocity \vec{Vₓ(t)}, a y-axis velocity \vec{V_{y}(t)}, and a z-axis velocity \vec{V_{z}(t)} at a current time t based on the initial velocity;
obtaining an x-axis displacement \vec{lₓ(t)}, a y-axis displacement \vec{l_{y}(t)}, and a z-axis displacement \vec{l_{z}(t)} based on the start time tₛₜₐᵣₜ, the current time t, the x-axis velocity \vec{Vₓ (t)}, the y-axis velocity \vec{V_{y}(t)}, and the z-axis velocity \vec{V_{z}(t)};
obtaining an actual displacement \vec{l(t)}: based on the x-axis displacement \vec{lₓ(t)}, the y-axis displacement \vec{l_{y}(t)}, and the z-axis displacement \vec{l_{z}(t)}; and
determining whether the actual displacement \vec{l(t)}: is zero, wherein when the actual displacement becomes zero, it indicates that one cycle of movement is completed, a current time is recorded as t_{end}, and the thrust count is incremented by 1.

4. The sexual health capacity measurement and analysis method according to claim 1, wherein said acquiring the thrust distance of the male, and/or the thrust distance of the female specifically comprises:
acquiring two consecutive sampling times, comprising a first sampling time tₙ and a second sampling time tₙ₊₁;
obtaining a change in velocity based on the first sampling time tₙ, the second sampling time tₙ₊₁, and an initial velocity at the first sampling time;
obtaining a velocity V_{x_n+1}, at the second sampling time based on the initial velocity at the first sampling time and the change in velocity; and
obtaining the thrust distance of the male and/or the thrust distance of the female based on the first sampling time tₙ, the second sampling time tₙ₊₁, the initial velocity at the first sampling time, and the velocity at the second sampling time.

5. The sexual health capacity measurement and analysis method according to claim 3, wherein said acquiring the thrust frequency of the male and/or the thrust frequency of the female specifically comprises:
obtaining the thrust frequency based on the thrust count.

6. The sexual health capacity measurement and analysis method according to claim 1, wherein said obtaining the position data based on the relative acceleration of the waist movements of the male and the female specifically comprises:
converting relative acceleration data of the waist movements of the male and the female into a vector in an object coordinate system; and obtaining the position data based on a relationship between the vector and a gravity vector;
or,
measuring an angular velocity by using a gyroscope, and integrating the angular velocity to obtain an object rotation angle at each time point; fusing the relative acceleration of the waist movements of the male and the female with the rotation angle using a Kalman filter to obtain fused data; and representing the fused data using quaternions to express the position data;
or,
identifying the relative acceleration of the waist movements of the male and the female by using a posture recognition model to obtain the position data.

7. The sexual health capacity measurement and analysis method according to claim 1, wherein construction of the hierarchical analysis model specifically comprises:
establishing a goal layer, a criterion layer, a first sub-criterion layer, a second sub-criterion layer, and a scheme layer, wherein the goal layer is used to predict a degree of premature ejaculation; the criterion layer is used to calculate the position data; the first sub-criterion layer is used to calculate the thrust count, thrust distance, and thrust frequency of the male, and/or the thrust count, thrust distance, and thrust frequency of the female; the second sub-criterion layer is used to calculate a distribution of the thrust frequency; and the scheme layer outputs sexual health capacity measurement and analysis results for different users;
constructing comparison matrices for the goal layer, the criterion layer, the first sub-criterion layer, the second sub-criterion layer, and the scheme layer, respectively;
performing hierarchical single sorting and a consistency test, as well as hierarchical total sorting and a consistency test on the comparison matrices;
calculating corresponding membership degrees of the comparison matrices using relative membership degrees;
obtaining a generalized weighted distance based on the corresponding membership degrees;
obtaining the sexual health capacity measurement and analysis results for different users based on the generalized weighted distance.

8. A sexual health capacity measurement and analysis system, comprising:
a data acquisition module configured to:
acquire a relative acceleration of waist movements of a male and a female;
acquire a start time and an end time of sexual activity to obtain a duration; and
acquire a thrust count, a thrust distance, and a thrust frequency of the male, and/or a thrust count, a thrust distance, and a thrust frequency of the female;
a posture recognition module connected to the data acquisition module and configured to obtain position data based on the relative acceleration of the waist movements of the male and the female; and
a hierarchical analysis module connected to the data acquisition module and the posture recognition module, and configured to input the relative acceleration of the waist movements of the male and the female, the duration, the thrust count, thrust distance, and thrust frequency of the male, and/or the thrust count, thrust distance, and thrust frequency of the female, and the position data into a hierarchical analysis model to obtain a sexual health capacity measurement and analysis result.

9. An electronic device, comprising a memory, a processor, and a computer program that is stored in the memory and executable by the processor, wherein the processor executes the computer program to implement the sexual health capacity measurement and analysis method according to claim 1.

10. A non-transitory computer storage medium storing a computer program, wherein when the computer program is executed, the sexual health capacity measurement and analysis method according to claim 1 is implemented.

11. The electronic device according to claim 9, wherein said acquiring the relative acceleration of the waist movements of the male and the female specifically comprises:
obtaining a waist acceleration of the male during movement by using an inertial measurement unit, wherein the waist acceleration of the male during movement comprises: a_{x_m}(t), a_{y_m}(t), and a_{z_m}(t);
obtaining a waist acceleration of the female during movement by using an inertial measurement unit, wherein the waist acceleration of the female during movement comprises: a_{x_w}(t), a_{y_w}(t), and a_{z_w}(t); and
obtaining the relative acceleration of the waist movements of the male and the female based on the waist acceleration of the male during movement and the waist acceleration of the female during movement, wherein the relative acceleration of the waist movements of the male and the female comprises: aₓ(t), a_{y}(t), and a_{z}(t); x, y, and z represent three directions of three-dimensional coordinate axes, a represents acceleration, m represents male, and w represents female;
$${a}_{x} ( t ) = {a}_{x \_ m} ( t ) - {a}_{x \_ w} ( t ) ;$$
$${a}_{y} ( t ) = {a}_{y \_ m} ( t ) - {a}_{y \_ w} ( t ) ;$$
$${a}_{z} ( t ) = {a}_{z \_ m} ( t ) - {a}_{z \_ w} ( t ) .$$

12. The electronic device according to claim 9, wherein said acquiring the thrust count of the male, and/or the thrust count of the female specifically comprises:
acquiring a start time tₛₜₐᵣₜ of a thrust, and obtaining an initial velocity;
obtaining an x-axis velocity \vec{Vₓ(t)}, a y-axis velocity \vec{V_{y}(t)}, and a z-axis velocity \vec{V_{z}(t)} at a current time t based on the initial velocity;
obtaining an x-axis displacement \vec{lₓ(t)}, a y-axis displacement \vec{l_{y}(t)}, and a z-axis displacement \vec{l_{z}(t)} based on the start time tₛₜₐᵣₜ, the current time t, the x-axis velocity \vec{Vₓ (t)}, the y-axis velocity \vec{V_{y}(t)}, and the z-axis velocity \vec{V_{z}(t)};
obtaining an actual displacement \vec{l(t)}: based on the x-axis displacement \vec{lₓ(t)}, the y-axis displacement \vec{l_{y}(t)}, and the z-axis displacement \vec{l_{z}(t)}; and
determining whether the actual displacement \vec{l(t)}: is zero, wherein when the actual displacement becomes zero, it indicates that one cycle of movement is completed, a current time is recorded as t_{end}, and the thrust count is incremented by 1.

13. The electronic device according to claim 9, wherein said acquiring the thrust distance of the male, and/or the thrust distance of the female specifically comprises:
acquiring two consecutive sampling times, comprising a first sampling time tₙ and a second sampling time tₙ₊₁;
obtaining a change in velocity based on the first sampling time tₙ, the second sampling time t₊₁, and an initial velocity at the first sampling time;
obtaining a velocity V_{x_n+1} at the second sampling time based on the initial velocity at the first sampling time and the change in velocity; and
obtaining the thrust distance of the male and/or the thrust distance of the female based on the first sampling time tₙ, the second sampling time tₙ₊₁, the initial velocity at the first sampling time, and the velocity at the second sampling time.

14. The electronic device according to claim 12, wherein said acquiring the thrust frequency of the male and/or the thrust frequency of the female specifically comprises:
obtaining the thrust frequency based on the thrust count.

15. The electronic device according to claim 9, wherein said obtaining the position data based on the relative acceleration of the waist movements of the male and the female specifically comprises:
converting relative acceleration data of the waist movements of the male and the female into a vector in an object coordinate system; and obtaining the position data based on a relationship between the vector and a gravity vector;
or,
measuring an angular velocity by using a gyroscope, and integrating the angular velocity to obtain an object rotation angle at each time point; fusing the relative acceleration of the waist movements of the male and the female with the rotation angle using a Kalman filter to obtain fused data; and representing the fused data using quaternions to express the position data;
or,
identifying the relative acceleration of the waist movements of the male and the female by using a posture recognition model to obtain the position data.

16. The electronic device according to claim 9, wherein construction of the hierarchical analysis model specifically comprises:
establishing a goal layer, a criterion layer, a first sub-criterion layer, a second sub-criterion layer, and a scheme layer, wherein the goal layer is used to predict a degree of premature ejaculation; the criterion layer is used to calculate the position data; the first sub-criterion layer is used to calculate the thrust count, thrust distance, and thrust frequency of the male, and/or the thrust count, thrust distance, and thrust frequency of the female; the second sub-criterion layer is used to calculate a distribution of the thrust frequency; and the scheme layer outputs sexual health capacity measurement and analysis results for different users;
constructing comparison matrices for the goal layer, the criterion layer, the first sub-criterion layer, the second sub-criterion layer, and the scheme layer, respectively;
performing hierarchical single sorting and a consistency test, as well as hierarchical total sorting and a consistency test on the comparison matrices;
calculating corresponding membership degrees of the comparison matrices using relative membership degrees;
obtaining a generalized weighted distance based on the corresponding membership degrees;
obtaining the sexual health capacity measurement and analysis results for different users based on the generalized weighted distance.

17. The non-transitory computer storage medium storing a computer program according to claim 10, wherein said acquiring the relative acceleration of the waist movements of the male and the female specifically comprises:
obtaining a waist acceleration of the male during movement by using an inertial measurement unit, wherein the waist acceleration of the male during movement comprises: \vec{a_{x_w}(t)}, \vec{a_{y_w}(t)}, and \vec{a_{z_w}(t)};
obtaining a waist acceleration of the female during movement by using an inertial measurement unit, wherein the waist acceleration of the female during movement comprises: a_{x_w}(t), a_{y_w}(t), and a_{z_w}(t); and
obtaining the relative acceleration of the waist movements of the male and the female based on the waist acceleration of the male during movement and the waist acceleration of the female during movement, wherein the relative acceleration of the waist movements of the male and the female comprises: aₓ(t), a_{y}(t), and a_{z} (t); x, y, and z represent three directions of three-dimensional coordinate axes, a represents acceleration, m represents male, and w represents female;
$${a}_{x} ( t ) = {a}_{x \_ m} ( t ) - {a}_{x \_ w} ( t ) ;$$
$${a}_{y} ( t ) = {a}_{y \_ m} ( t ) - {a}_{y \_ w} ( t ) ;$$
$${a}_{z} ( t ) = {a}_{z \_ m} ( t ) - {a}_{z \_ w} ( t ) .$$

18. The non-transitory computer storage medium storing a computer program according to claim 10, wherein said acquiring the thrust count of the male, and/or the thrust count of the female specifically comprises:
acquiring a start time tₛₜₐᵣₜ of a thrust, and obtaining an initial velocity;
obtaining an x-axis velocity \vec{Vₓ(t)}, a y-axis velocity \vec{V_{y}(t)}, and a z-axis velocity \vec{V_{z}(t)} at a current time t based on the initial velocity;
obtaining an x-axis displacement \vec{lₓ(t)}, a y-axis displacement \vec{l_{y}(t)}, and a z-axis displacement \vec{l_{z}(t)} based on the start time tₛₜₐᵣₜ, the current time t, the x-axis velocity \vec{Vₓ(t)}, the y-axis velocity \vec{V_{y}(t)}, and the z-axis velocity \vec{V_{z}(t)};
obtaining an actual displacement \vec{l(t)}: based on the x-axis displacement \vec{lₓ(t)}, the y-axis displacement \vec{l_{y}(t)}, and the z-axis displacement \vec{l_{z}(t)}; and
determining whether the actual displacement \vec{l(t)}: is zero, wherein when the actual displacement becomes zero, it indicates that one cycle of movement is completed, a current time is recorded as t_{end}, and the thrust count is incremented by 1.

19. The non-transitory computer storage medium storing a computer program according to claim 10, wherein said acquiring the thrust distance of the male, and/or the thrust distance of the female specifically comprises:
acquiring two consecutive sampling times, comprising a first sampling time tₙ and a second sampling time tₙ₊₁;
obtaining a change in velocity based on the first sampling time tₙ, the second sampling time tₙ₊₁, and an initial velocity at the first sampling time;
obtaining a velocity V_{x_n+1} at the second sampling time based on the initial velocity at the first sampling time and the change in velocity; and
obtaining the thrust distance of the male and/or the thrust distance of the female based on the first sampling time tₙ, the second sampling time tₙ₊₁, the initial velocity at the first sampling time, and the velocity at the second sampling time.

20. The non-transitory computer storage medium storing a computer program according to claim 18, wherein said acquiring the thrust frequency of the male and/or the thrust frequency of the female specifically comprises:
obtaining the thrust frequency based on the thrust count.
